(19) United States
(12) Patent Application Publication
(10) Pub. No.: US 2008/0152730 A1
(43) Pub. Date: Jun. 26, 2008

(54) Novel Composition Comprising Ligustilide and Process For Their Manufacture

(76) Inventors: Ralf Proplesch, Wolflinswil (CH); Christoph Riegger, Bettingen (CH); Werner Simon, Riehen (CH); Swen Wolfram, Waldshut-Tiengen (DE)

Correspondence Address: Stephen M. Haracz;Bryan Cave, 1290 Avenue of the Americas, New York, NY, 10104, US

(21) Appl. No.: 11/883,658

(22) PCT Filed: Jan. 26, 2006
(86) PCT No.: PCT/EP06/00648

§ 371 Date: Aug. 2, 2007

(30) Foreign Application Priority Data
Feb. 4, 2005 (EP) 05002333.2

(51) Int. Cl. A61K 36/23

(52) U.S. Cl. 424/725

(57) ABSTRACT

Novel purified extracts from Ligusticum species are obtained by submitting crude extracts from Ligusticum species, particularly from Ligusticum wallichii (chuanxiong) containing less than 50 wt.-% of ligustilide to rectification under specific conditions.

The present invention relates to a novel composition comprising ingredients from Ligusticum species, especially L. wallichii and to a process for the manufacture thereof. More particularly, the present invention relates to a purified extract from Ligusticum species comprising at least about 50 wt.-% of ligustilide, and no more than 5 wt.-% of fatty acids and glycerides.

Extracts from Ligusticum species such as L. wallichii (also known as L. Chuanxiong) are used in traditional Chinese, Japanese and Korean medicine for the treatment of various ailments, e.g., headache, abdominal pain, menstrual disorders and rheumatic arthralgia. Commercially available extracts, e.g. as obtained by supercritical fluid extraction are dark brown, strongly smelling products containing typically up to about 40 wt.-% e.g., 30-40 wt.-%, of phthalides as the essential physiologically active ingredients, particularly ligustilide, senkyunolide, sedanolide, 3-n-butylphthalide and 3-butylidenephthalide, and fatty acids, glycerides and some water. The dark colour and the intense smell of these extracts render them less suitable for use as medicaments and, especially, as nutritional supplements.

In accordance with the present invention it has been found that commercial extracts from Ligusticum species can be improved as to their organoleptic properties and appearance while enriching the essential physiologically active ingredients, especially ligustilide, therein.

Thus, in one aspect, the present invention is concerned with a purified and enriched extract from Ligusticum species, especially L. wallichii, comprising at least about 50 wt.-%, particularly about 50-60 wt.-% of ligustilide, and no more than 5 wt.-% of fatty acids and glycerides. In a particular embodiment the invention is concerned with a purified and enriched extract from Ligusticum species, especially L. wallichii, comprising at least about 50 wt.-% of ligustilide, and no more than 10 wt.-% of fatty acids and triglycerides.

In another aspect, the present invention is concerned with a purified and enriched extract from Ligusticum species, especially L. wallichii, comprising at least about 85 wt.-%, especially at least 90 wt.-% of phthalides found in Ligusticum species.

In still another aspect, the present invention is concerned with a purified and enriched extract from Ligusticum species, especially L. wallichii, comprising about 50 to 60 wt.-% of ligustilide, and no more than 5 wt.-% of fatty acids and glycerides.

A preferred purified extract in accordance with the present invention comprises 50 to 60 wt-% of ligustilide, at least 20 wt.-% of senkyunolide, 1 to 5 wt.-% of 3-butylidenephthalide, 1-5 wt.-% of 3-n-butylphthalide, 1-5 wt.-% of sedanolide and no more than 5 wt.-% of fatty acids and glycerides. Such preferred purified extracts have a color of below 9, especially of no more than 6, on the Gardner scale.

In still another aspect, the present invention is concerned with a process for the manufacture of the purified and enriched extracts from Ligusticum species as defined above.

According to that aspect of the invention, an extract of Ligusticum species containing less than 50 wt.-% of ligustilide and more than 5 wt-% of fatty acids and glycerides is submitted to a rectification. The rectification is suitably carried out at a temperature of about of 130° C. to about 400° C., and at pressure of about 0.1 mbar to about 25 mbar. In a preferred embodiment, the rectification is carried out at a heating temperature of about 200° C. to about 230° C. and a top pressure of the rectification column of about 0.1 mbar to about 3 mbar. Suitably, the extract used as the starting material in the process of this invention is an extract from roots of Ligusticum species, especially dried roots from L. wallichii and is obtained by supercritical fluid extraction using, e.g., carbon dioxide. In a preferred embodiment, the extract, prior to rectification is submitted to degassing in a degassing unit. The degassing unit may be any evaporating system that allows to remove water from the extract by applying heat and reduced pressure. Conveniently, the degassing is carried out at a temperature of from 120-180° C. at 10-50 mbar.

By the process of the present invention ligustilide and other phthalides like senkyunolide, 3-n-butylphthalide, sedanolide, 3-butylidenephthalide can be enriched in the resulting distillate to over 90% based on the weight of the distillate. In contrast to the starting material, the distillate obtained in accordance with the invention smells pleasantly and shows a light yellow colour. Glycerides and free fatty acids are enriched in the distillation residue.

The rectification can be performed with all kind of evaporator types, however a preferred equipment is a wiped thin film evaporator with a short residence time, preferably not exceeding 3 minutes and low pressure drop.

The rectification column can be equipped with all kind of different column internals like trays, random or structured packings, however a preferred internal is a structured packing with a low pressure drop and a small liquid hold up. This prevents the degradation of the phthalides at higher temperatures and longer residence times.

A preferred rectification column set up in accordance with the present invention is equipped with a liquid side draw in the rectifying section of the column. If the resulting purified ligusticum extract is taken out of the rectification column as a liquid side draw, it leads to a higher phthalide concentration because other light boiling components compared to the phthalides can be separated with the distillate stream. The same effect can be archieved if the rectification is equipped with a divided wall column. In this case the resulting purified ligusticum extract is also taken out of the column as a side draw.

The process of the invention can be carried out batchwise and preferred in continuous mode due to the thermal instability of the phthalides. The purified extract as obtained by the process of the present invention can be converted into solid formulations by conventional techniques.

The novel extract of this invention can be formulated, in liquid or solid form, into nutraceutical compositions such as food items, or nutritional supplements which may contain further nutritionally desirable ingredients such as vitamins, minerals and trace elements. The nutraceutical compositions may contain the extract of the present invention in an amount sufficient to provide a dosage of from 0.01-50 mg ligustilide per kg body weight of the subject in need of ligustilide administration per day.

The following Example illustrate the invention further.

### EXAMPLE 1

Crude ligusticum extract, e.g., as obtained by supercritical extraction with carbon dioxide from Ligusticum roots with a total phthalide concentration of 29 wt.-% (8.2 wt.-% senkyunolide, 0.5 wt.-% 3-n-butylphthalide, 1.2 wt.-% sedanolide, 18.3 ligustilide, 0.6 wt.-% 3-butylidenephthalide) was purified by a continuous vacuum rectification to a total phthalide concentration of 90 wt.-% (26.4 wt.-% senkyunolide, 1.6 wt.-% 3-n-butylphthalide, 3.7 wt.-% sedanolide, 56.3 wt.-% ligustilide, 2.0 wt.-% 3-butylidenephthalide).

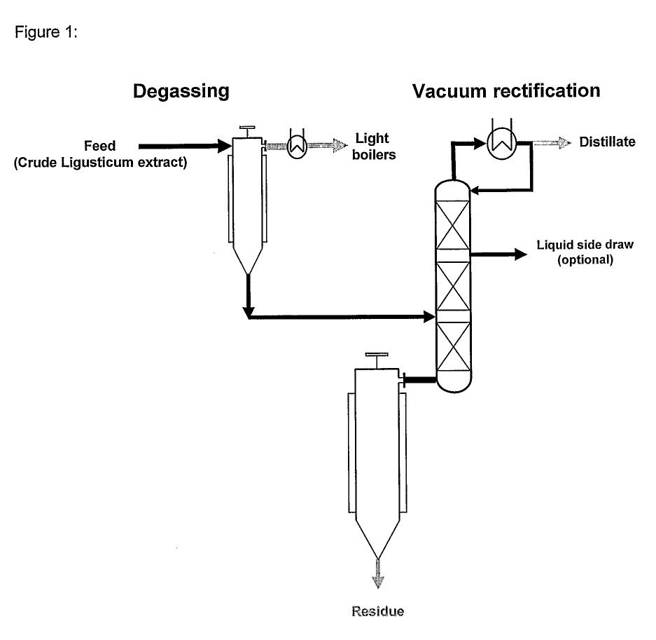

At first the crude ligusticum was degassed in order to separate the water from the crude extract. At a reduced pressure of 25 mbar and a heating temperature of 160° C. approx. 1.0% of the feed amount was evaporated. The residue comprises the crude ligusticum extract almost free off water. This material was fed continuously into the rectification setup (wiped thin film evaporator with a heating area 0.05 m², distillation column with 1 m height structured packings) in order to concentrate the phthalides in the resulting distillate stream of the column (see FIG. 1 with liquid side draw not operating). At conditions with a reduced top column pressure of 0.5 mbar and at a heating temperature of 230° C. the distillate/feed-ratio was 0.33:1. The reflux ratio of the distillate stream was about 1. Glycerides and free fatty acids are enriched in the distillation residue. The distillate stream contains all above mentioned phthalides in a total phthalide concentration of 90 wt.-%. The colour of the final purified ligusticum extract was 4.7 on Gardner scale.

### EXAMPLE 2

Crude ligusticum extract, e.g., as obtained by supercritical extraction with carbon dioxide from Ligusticum roots with a total phthalide concentration of 36 wt.-% (11.4 wt.-% senkyunolide, 1.1 wt.-% 3-n-butylphthalide, 1.6 wt.-% sedanolide, 20.4 ligustilide, 1.3 wt.-% 3-butylidenephthalide) was purified by a continuous vacuum rectification with a liquid side draw to a total phthalide concentration of 94 wt.-% (29.3 wt.-% senkyunolide, 3.3 wt.-% 3-n-butylphthalide, 3.9 wt.-% sedanolide, 53.5 wt.-% ligustilide, 3.9 wt.-% 3-butylidene-phthalide) in the following way.

At first the crude ligusticum was degassed in order to separate the water from the crude extract. At a reduced pressure of 25 mbar and a heating temperature of 160° C. approx. 1.0% of the feed amount was evaporated. The residue comprises the crude ligusticum extract almost free off water. This material was fed continuously into the rectification setup (wiped thin film evaporator with a heating area 0.05 m², distillation column with 1.5 m height structured packings, liquid side draw at column height 1 m from below) in order to concentrate the phthalides in the resulting liquid side stream (see FIG. 1 with liquid side draw operating).

At a reduced pressure of 1 mbar and a heating temperature of 230° the feed stream was separated as follows into 36% liquid side stream, 62% residue and 2% distillate. The reflux ratio of the distillate was about 10 and the refluxratio of the side stream was about 1. Glycerides and free fatty acids are enriched in the distillation residue and the light boiling components are enriched in the distillate. The desired phthalides are enriched in the liquid side stream with a concentration of 94%. The colour of the the final purified ligusticum extract was 4.6 on Gardner scale.

### EXAMPLE 3

Tablets are prepared by conventional procedures containing, as active ingredient, a granulate prepared from an extract as obtained in Example 1 or 2 in an amount providing 50 mg of ligustilide per tablet, and excipients (microcrystalline cellulose, silicone dioxide (SiO2), magnesium stearate, crosscarmellose sodium) ad 200 mg.

### EXAMPLE 4

A Soft Drink containing an ligustilide extract may be prepared as follows:

1. A Soft Drink Compound is prepared from the following ingredients:

| Juice concentrates and water soluble flavours | [g] |
|---|---|
| Orange concentrate | |
| 60.3° Brix, 5.15% acidity | 657.99 |
| Lemon concentrate | |
| 43.5° Brix, 32.7% acidity | 95.96 |
| Orange flavour, water soluble | 3.43 |
| Apricot flavour, water soluble | 6.71 |
| Water | 26.46 |

1.2 Color

| | |
|---|---|
| β-Carotene 10% CWS | 0.89 |
| Water | 67.65 |

1.3 Acid and Antioxidant

| | |
|---|---|
| Ascorbic acid | 4.11 |
| Citric acid anhydrous | 0.69 |
| Water | 43.18 |

1.4 Stabilizers

| | |
|---|---|
| Pectin | 0.20 |
| Sodium benzoate | 2.74 |
| Water | 65.60 |

1.5 Oil Soluble Flavours

| | |
|---|---|
| Orange flavour, oil soluble | 0.34 |
| Orange oil distilled | 0.34 |

1.6 Active Ingredient

Ligustilide extract as obtained in Example 1 or 2 in an amount providing 500 mg ligustilide

Fruit juice concentrates and water soluble flavours are mixed without incorporation of air. The color is dissolved in deionized water. Ascorbic acid and citric acid is dissolved in water. Sodium benzoate is dissolved in water. The pectin is added unter stirring and dissolved while boiling. The solution is cooled down. Orange oil and oil soluble flavours are premixed. The active ingredient as mentioned under 1.6 is stirred into the fruit juice concentrate mixture (1.1).

In order to prepare the soft drink compound all parts 3.1.1 to 3.1.6 are mixed together before homogenizing using a Turrax and then a high-pressure homogenizer (p₁=200 bar, p₂=50 bar).

1. A purified extract from Ligusticum species comprising at least about 50 wt.-% of ligustilide and no more than about 5 wt.-% of fatty acids and glycerides.

2. An extract as in claim 1 comprising at least about 50 wt.-% of ligustilide and no more than about 10 wt.-% of fatty acids and triglycerides.

3. An extract as in claim 1 comprising at least about 70 wt.-% of ligustilide.

4. An extract as in claim 1 comprising at least about 85 wt.-% of phthalides found in Ligusticum species.

5. An extract as in claim 1 comprising at least about 90 wt.-% of phthalides found in Ligusticum species.

6. An extract as in claim 1 comprising about 50 to 60 wt.-% of ligustilide.

7. An extract as in claim 6 comprising 50 to 60 wt-% of ligustilide, at least 20 wt.-% of senkyunolide, 1 to 5 wt.-% of 3-butylidenephthalide, 1-5 wt.-% of 3-n-butylphthalide and 1-5 wt.-% of sedanolide.

8. An extract as in claim 1 which is from Ligusticum wallichii (chuanxiong).

9. An extract as in claim 1 which has a color of below 9, especially of no more than 6, on the Gardner scale.

10. A process for the preparation of an extract as defined in claim 1 which comprises submitting a crude extract from Ligusticum species, particularly from Ligusticum wallichii (chuanxiong), containing less than 50 wt.-% of ligustilide to rectification under reduced pressure and at a temperature of from about 130° C. to about 400° C., and collecting the distillate.

11. A process as in claim 10 wherein the rectification is carried out at a temperature of about of 130° C. to about 400° C. and pressure of about 0.1 to about 25 mbar, preferably at a temperature of about 200° C. to about 230° C., and a top pressure of the rectification column of about 0.1 to about 5 mbar.

12. A process as in claim 1 wherein the crude extract is degassed prior to rectification.

13. A process as in claim 11 wherein degassing is accomplished by heating the extract to about 120° C. to about 180° C.

14. A process as in claim 10 wherein the extract is obtained by supercritical fluid extraction of roots from Ligusticum species.

15. A process as in claim 10 wherein the distillate is converted into a dry composition.

16. A nutraceutical composition comprising an extract obtained according to claim 1.

17. (canceled)
